(19) United States
(12) Patent Application Publication
(10) Pub. No.: US 2008/0248536 A1
(43) Pub. Date: Oct. 9, 2008

(54) Polynucleotide Ligation Reactions

(75) Inventor: Preben Lexow, Oslo (NO)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LINGVITAE AS, Oslo (NO)

(21) Appl. No.: 10/586,560

(22) PCT Filed: Jan. 21, 2005
(86) PCT No.: PCT/GB05/00225

§ 371 Date: Jan. 7, 2008

Related U.S. Application Data
(60) Provisional application No. 60/560,336, filed on Apr. 6, 2004.

(30) Foreign Application Priority Data
Jan. 23, 2004 (GB) 0401525.1

(51) Int. Cl. C12P 19/34; C07H 1/00

(52) U.S. Cl. 435/91.52; 536/124

(57) ABSTRACT

The method of the invention improves the specificity of a ligation reaction carried out between a first double-stranded polynucleotide having a single-stranded portion and a second polynucleotide having a complementary single-stranded portion, the second polynucleotide being present in a sample comprising a mixture of different polynucleotides. The method comprises contacting the sample, under hybridising conditions, with the first polynucleotide and one or more third polynucleotide(s) wherein the third polynucleotide(s) comprises a single-stranded portion that differs from the single-stranded portion of the first polynucleotide by at least one base substitution.

### FIELD OF INVENTION

The present invention relates to methods for improving polynucleotide ligation reactions.

### BACKGROUND TO INVENTION

Berg and Boyer created the first recombinant DNA molecule in 1972. This simple concept of recombination—the splicing together of two pieces of DNA and fusing them by ligation, is the basis for the entire field of molecular biology. Molecular biology has become ubiquitous to the point where it is central to the majority of all biological research. The ligation reaction is performed thousands of times a day in research and diagnostic laboratories worldwide. Given the boundless opportunity presented by genetic engineering, the ligation reaction is likely to remain a central technique for many years to come.

The ligation reaction itself is chemically simple, comprising the linking of two nucleotides by the creation of a phosphodiester bond between the 3′ hydroxyl of one nucleotide and the 5′ phosphate of another, by a ligase enzyme. There are two types of ligation, known as “sticky end” and “blunt end”, depending on the presence or lack (respectively) of complementary single stranded regions on the two polynucleotides to be joined, in proximity to the ligation location. “Sticky-end” ligations involve the hybridisation of complementary single stranded sequences between the two polynucleotides to be joined, prior to the ligation event itself. Sequences that have similar but not 100% complementary single stranded sequences will still be ligated, known as a mismatch ligation. These result in the ligation of an incorrect sequence and decrease the efficiency and fidelity of the overall ligation reaction.

Since ligation is such an important reaction, ligases are available on the market that are improved, modified and optimised to give maximum efficiency. These enzymes are expensive and it is therefore desirable to use as a small amount as is possible without reducing the efficiency of the reaction and whilst avoiding mismatch ligation. Mismatch ligations are problematic as they are deleterious to the fidelity of the ligation process. It is therefore desirable to minimise mismatch ligations.

Current methods of increasing ligation specificity include decreasing the amount of ligase and increasing the salt in the reaction mix to slow down the reaction. Since match ligations are much faster than mismatch ligations, the increased specificity observed using this technique is a result of the slower reaction speed and whilst this increases the match:mismatch ratio, it results in a low yield and does not prevent mismatch ligations.

There is therefore a need for improvements in ligation reactions.

### SUMMARY OF INVENTION

The present invention is based on the realisation that specificity in “sticky-end” ligations can be increased by including short adapters that reduce the occurrence of mismatch ligation.

According to a first aspect of the invention, a method for improving the specificity of a ligation reaction carried out between a first double stranded polynucleotide having a single stranded portion and a second polynucleotide having a complementary single stranded portion, said second polynucleotide being present in a sample comprising a mixture of different polynucleotides, comprises:

contacting the sample, under hybridising conditions, with the first polynucleotide and one or more third polynucleotide(s), wherein the third polynucleotide(s) comprises a single stranded portion that differs from the single stranded portion of the first polynucleotide by at least one base substitution, and carrying out a ligation reaction.

The present invention improves the yield of match ligations by reducing mismatch ligations through the use of blocking polynucleotides which hybridise to incorrect single stranded overhangs on the second polynucleotides.

### DESCRIPTION OF THE DRAWINGS

The present invention is illustrated by reference to the accompanying drawing, where:

FIG. 1 is a graphic illustration of match:mismatch ratio as a function of time, wherein FIG. 1a illustrates the ratio in the absence of blocking adapters, and FIG. 1b illustrates the ratio in the presence of blocking adapters.

### DETAILED DESCRIPTION OF THE INVENTION

The present invention is used to increase specificity of polynucleotide ligations. The term “polynucleotide” is used herein to refer to biological molecules made up of a plurality of nucleotides. Preferred polynucleotides include DNA, RNA and synthetic analogues thereof, including PNA.

The term “hybridising conditions” is used herein to refer to conditions that allow complementary base pairing to occur between two polynucleotides, such that two complementary single stranded polynucleotides will hybridise to form a duplex. Such conditions are well known in the art. An Example of such conditions is incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. Alternative conditions will be apparent to the skilled person and are applicable to the present invention.

In any ligation reaction, two polynucleotide molecules are joined. The term “first polynucleotide” is used herein to refer to one of the two intended targets of ligation. The term “second polynucleotide” is used herein to refer to the other of the two intended targets of ligation.

A non-limiting example of the terms “first polynucleotide” and “second polynucleotide” comprises the “first polynucleotide” being a DNA vector into which an insert, the “second polynucleotide”, is to be ligated to form a recombinant construct.

In any ligation reaction, there may be polynucleotides present which are neither “first polynucleotides” or “second polynucleotides”, in the sense that they are not intended to be part of the ligation reaction. These polynucleotides interfere with the ligation between the first and second polynucleotides, which results in mismatch ligations.

As used herein, the term “third polynucleotide” is used to describe polynucleotides which are added to the ligation reaction mixture to hybridise to any polynucleotide which is not a first or second polynucleotide, preventing the unwanted polynucleotides from reacting with the other components of the reaction mix. The third polynucleotides are not totally complementary to the first or second polynucleotides.

The method increases specificity in polynucleotide ligations through the addition of one or more third polynucleotide(s) into a reaction mix. This reaction mix comprises a first polynucleotide and a second polynucleotide, which contain complementary single stranded portions. The second polynucleotide is present in a sample comprising a mixture of different polynucleotides, and is the intended target for binding to the first polynucleotide. The third polynucleotide(s) comprises at least a single stranded portion that differs from the single stranded portion of the first polynucleotide by at least one base. The number of differences between the first and third polynucleotides may depend on the size of the single stranded portions involved. For example, if the single stranded portion is only 3 bases in length, a single difference may be suitable, but if the single stranded portion is 6 bases in length, multiple differences may be preferred. The differences may be substitution(s), deletion(s) or addition(s).

The third polynucleotide may be added to the sample containing the second polynucleotide simultaneously with or sequentially before or after the first polynucleotide. The third polynucleotide is preferably added to the sample containing the second polynucleotide, along with the first polynucleotide.

Preferably, the third polynucleotide is present in excess with respect to the first and second polynucleotides, to ensure that all other polynucleotides in the sample are hybridised by the third polynucleotide.

It is intended that the first and second polynucleotides hybridise and are ligated together, to the exclusion of other polynucleotides in the sample. The third polynucleotide(s) hybridise to the other polynucleotides in the sample which would otherwise compete for binding to the first polynucleotides, effectively preventing them from hybridising to the first polynucleotides and increasing the number of correct binding events between the first and second polynucleotides. This increases the specificity of the overall ligation reaction.

Preferably the mixture of third polynucleotides comprises double stranded polynucleotides with a single stranded portion, such that the single stranded portion hybridises its complementary region on incorrect first and second target polymers.

Preferably, the single stranded portion of each of the first, second and third polynucleotides is from 3 to 6 bases in length. Most preferably, the single stranded portion is 4 bases in length.

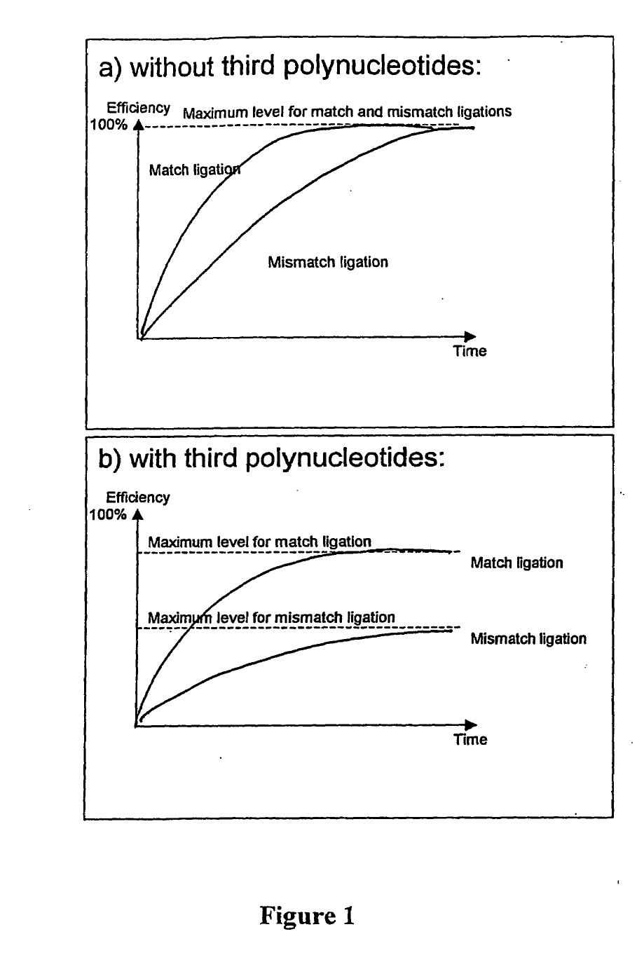

FIG. 1 is a graphical representation of the match:mismatch ratio as a function of time. This ratio becomes lower as the reaction progresses, since the match reaction rapidly reaches plateau and is caught up by the slower mismatch reaction. Traditional methods of increasing specificity merely slow the reaction (using less ligase or increasing salt concentration) and shift the reaction to the left of the graph, where the match:mismatch ratio is favourable but yield is decreased. The present invention ensures that the mismatch ligations do not increase. The match reaction can proceed to full term without the mismatch reaction ever catching up. This provides an optimised match:mismatch ratio.

The invention will now be illustrated with reference to the following, non-limiting, example.

### EXAMPLE

A test system was set up to measure the effect on ligation specificity of adding blocking adapters to a ligation reaction. The goal of this test system was to measure ligation specificity, i.e. the percentage of correctly ligated molecules relative to the total number of molecules ligated. The test system used makes use of the fact that a ligation product containing a single base mismatch in the ligation overhang region differs from correctly ligated ligation product by only one base. The protocol used was developed to measure ligation specificity (match ligation as percentage of total ligation) using the Homogeneous Mass Extend method (Sequenom) and the MassARRAY system (Sequenom). The protocol was used to measure the specificity of ligation for the 3′-most base (upper strand) of the ligation overhang region.

The polynucleotides used in this Example are shown in Table 1.

TABLE I
Sequences of adapters used

DPA 12

(SEQ ID NO: 1)

5′-TGTGTCCGCGTGGCTCTTCTATTCTTGGCTTTTCGTCGCTTGGCTTT
TCGTCGCTTGGTCATTCGTCGCTTGGTCATTCGTCGCTTGGCTTTTCGTC
GCTTGGTCATTCGTCG-3′

(SEQ ID NO: 2)

3′-ACACAGGCGCACCGAGAAGATAAGAACCGAAAAGCAGCGAACCGAAA
AGCAGCGAACCAGTAAGCAGCGAACCAGTAAGCAGCGAACCGAAAAGCAG
CGAACCAGTAAGCAGCTAA-5′
DPA 14

(SEQ ID NO: 3)

5′-TGTGTCCGCGTGGCTCTTCTATTCTTGGCTTTTCGTCGCTTGGCTTT
TCGTCGCTTGGTCATTCGTCGCTTGGTCATTCGTCGCTTGGTCATTCGTC
GCTTGGCTTTTCGTCG-3′

(SEQ ID NO: 4)

3′-ACACAGGCGCACCGAGAAGATAAGAACCGAAAAGCAGCGAACCGAAA
AGCAGCGAACCAGTAAGCAGCGAACCAGTAAGCAGCGAACCAGTAAGCAG
CGAACCGAAAAGCAGCTAA-5′
DPA 24

(SEQ ID NO: 5)

5′-TGTGTCCGCGTGGCTCTTCTATTCTTGGCTTTTCGTCGCTTGGTCAT
TCGTCGCTTGGTCATTCGTCGCTTGGCTTTTCGTCGCTTGGCTTTTCGTC
GCTTGGCTTTTCGTCG-3′

(SEQ ID NO: 6)

3′-ACACAGGCGCACCGAGAAGATAAGAACCGAAAAGCAGCGAACCAGTA
AGCAGCGAACCAGTAAGCAGCGAACCGAAAAGCAGCGAACCGAAAAGCAG
CGAACCGAAAAGCAGCTAA-5′
DPA 44

(SEQ ID NO: 7)

5′-GTGTCCGCGTGGCTCTTCTATTCTTGGTCATTCGTCGCTTGGCTTTT
CGTCGCTTGGTCATTCGTCGCTTGGTCATTCGTCGCTTGGCTTTTCGTCG
CTTGGTCATTCGTCG-3′

(SEQ ID NO: 8)

3′-ACACAGGCGCACCGAGAAGATAAGAACCAGTAAGCAGCGAACCGAAA
AGCAGCGAACCAGTAAGCAGCGAACCAGTAAGCAGCGAACCGAAAAGCAG
CGAACCAGTAAGCAGCTAA-5′
Target
5′-ATTTATCTGCTGCATGATCCGATAGTGCGAAT-3′

(SEQ ID NO: 9)

3′-ATAGACGACGTACTAGGCTATCACGCTTANNNN-5′
SLA 13
5′-NATCTAGATGCACTCCCGGACCTC-3′

(SEQ ID NO: 10)

3′-ATCTACGTGAGGGCCTGGAG-5′
SLA 14
5′-NATGTAGATGCACTCCCGGACCTC-3′

(SEQ ID NO: 11)

3′-ATCTACGTGAGGGCCTGGAG-5′
SLA 24
5′-NCGATAGATGCACTCCCGGACCTC-3′

(SEQ ID NO: 12)

3′-ATCTACGTGAGGGCCTGGAG-5′
SLA 44
5′-NGTATAGATGCACTCCCGGACCTC-3′

(SEQ ID NO: 13)

3′-ATCTACGTGAGGGCCTGGAG-5′
BLA 13-1
5′-NBTCATGAGCTGGGCGGCACGTAG-3′

(SEQ ID NO: 14)

3′-TACTCGACCCGCCGTGCATA-5′
BLA 13-2
5′-NAVCATGAGCTGGGCGGCACGTAG-3′

(SEQ ID NO: 15)

3′-TACTCGACCCGCCGTGCATA-5′
BLA 13-3
5′-NATDATGAGCTGGGCGGCACGTAG-3′

(SEQ ID NO: 16)

3′-TACTCGACCCGCCGTGCATA-5′
In Table I, “N” represents any of the bases G, C, T and A; “B” represents any of the bases G, C and T; “D” represents any of the bases A, C and T; “H” represents any of the bases A, C and T; and “V” represents any of the basese A, G and C.

Reactions were carried out where three of the following adapters were ligated together (see Table 1):

### 1) a Design Polymer Adapter (DPA)

2) a target molecule with a 4 nt 5′ overhang representing all 256 possible permutations of 4 nucleotides (‘NNNN’)
3) a Specific Ligation Adapter (SLA) meant to ligate specifically to a subset ( 1/64^{th}) of the target molecules.
Based on its 4 nt overhang, the SLA was supposed to ligate to only 4 out of the 256 permutations of the target.

There were four variants of each DPA (designated 13, 14, 24 and 44) and four of each SLA (designated similarly), see Table I. In some cases, a specific set of three Blocking Adapters (BLA's) were added to each ligation reaction to block all nine single base mismatches possible for a particular reaction (see Table I); there were 3×4 variants of each BLA (Table I).

For each reaction, DPA, Target and the corresponding SLA were ligated (at a molecular ratio of DPA:Target:SLA=64:64:10) in the presence or absence of the specific set of nine BLA's (each BLA was at an equimolar ratio relative to the SLA) using T4 DNA ligase (Fermentas). PCR using the primers described in Table II was performed on 1/20^{th} of the ligation reaction to amplify the ligation products.

TABLE II
PCR and Extension primers (in 5′ to 3′ orientation)

| | | |
|---|---|---|
| Forward PCR primer | ACGTTGGATGTGTGTCCGCGTGGCTCTTCT | (SEQ ID NO: 17) |
| Reverse PCR primer | ACGTTGGATGATGGGCTTTTGAGGTCCGGGAGTG | (SEQ ID NO 18) |
| Extension primer | GAGGTCCGGGAGTGCATCTA | (SEQ ID NO: 19) |

Targets ligated to BLAs could not be amplified during this PCR due to the different sequence at their 3′ end (upper strand). PCR products were isolated and concentrated using the MinElute PCR Cleanup Kit (Qiagen).

The cleaned, concentrated PCR products were used in an extension reaction following the Homogenous Mass Extend protocol described in the Mass ARRAY User's Manuals (Sequenom). Basically, the reaction consisted of the product of the PCR amplified ligation product, an extension primer complementary to the sequence 5′ of the base to be investigated (see Table II), a ‘Stop mix’ (a specific mixture of equimolar amounts of one NTP in the dNTP form and the remaining three NTPs in the ddNTPs form), a thermostable polymerase in 1× buffer (all components from Sequenom except: ddNTPs from Roche, dNTPs from Amersham). The stop mix was chosen so that a base at the most 3′ end of the ligation overhang region resulting from a correct (match) ligation would yield a 2-base extension product whereas a base resulting from a mismatch ligation would yield a 1-base extension product.

Extension reactions and the subsequent washes were performed following the Homogenous Mass Extend method (Sequenom). Extension products were spotted on a SpectroCHIP (Sequenom) and separated according to mass as described in the Mass ARRAY User's Manuals (Sequenom). The results were a set of peaks representing unextended extension primer and (1-base and 2-base) extension products. The intensities of these peaks were transferred manually to a spreadsheet. The relative intensities of the 2-base extension product (representing the match ligation extension product) versus the total intensity of the extension products was calculated.

The results of the experiments are shown in Table III.

TABLE III
Percentage match ligation extension product of the total
extension product for the 3′-most base of the overhang

| Reaction | Without Blocking | With Blocking |
|---|---|---|
| (overhang) | Adapters | Adapters |
| 13 (NATC) | 84.4% | 88.0% |
| 14 (NATG) | 100.0% | 100.0% |
| 24 (NCGA) | 100.0% | 100.0% |
| 44 (NGTA) | 75.8% | 84.4% |

These results indicate that two out of the four ligation reactions (numbers 14 and 24) did not produce a detectable peak for a mismatch ligation product at the 3′-most position. For the other two reactions (13 and 44) the peak intensities for the match-ligation extension product detected represented 84.4% (reaction 13) and 75.8% (reaction 44) of the total extension product intensity, respectively, in the reactions without Blocking Adapters. These intensities were higher when Blocking Adapters were included in the reactions: 88.0% (reaction 13) and 84.4% (reaction 44). This indicates that blocking adapters helped to reduce mismatch ligations occurring at the 3′-most position.

1. A method for improving the specificity of a ligation reaction carried out between a first double stranded polynucleotide having a single stranded portion and a second polynucleotide having a complementary single stranded portion, said second polynucleotide being present in a sample comprising a mixture of different polynucleotides, comprising:
contacting the sample, under hybridising conditions, with the first polynucleotide and one or more third polynucleotide(s), wherein the third polynucleotide(s) comprises a single stranded portion that differs from the single stranded portion of the first polynucleotide by at least one base substitution, and carrying out a ligation reaction.

2. A method according to claim 1, wherein the third polynucleotide is a double stranded polynucleotide having said single stranded portion.

3. A method according to claim 1, wherein the single stranded portion on each of the first, second and third polynucleotides is from 3 to 6 bases in length.

4. A method according to claim 3, wherein the single stranded portion is 4 bases in length.

5. A method according to, claim 1 wherein the single stranded portion of the third polynucleotide differs from the single stranded portion of the first polynucleotide by one base.

6. A method according to claim 1, wherein the ligase is T4 DNA ligase.

7. A method according to claim 2, wherein the single stranded portion on each of the first, second and third polynucleotides is from 3 to 6 bases in length.

8. A method according to claim 7, wherein the single stranded portion is 4 bases in length.

9. A method according to claim 2 wherein the single stranded portion of the third polynucleotide differs from the single stranded portion of the first polynucleotide by one base.

10. A method according to claim 3 wherein the single stranded portion of the third polynucleotide differs from the single stranded portion of the first polynucleotide by one base.

11. A method according to claim 4 wherein the single stranded portion of the third polynucleotide differs from the single stranded portion of the first polynucleotide by one base.

12. A method according to claim 7 wherein the single stranded portion of the third polynucleotide differs from the single stranded portion of the first polynucleotide by one base.

13. A method according to claim 8 wherein the single stranded portion of the third polynucleotide differs from the single stranded portion of the first polynucleotide by one base.

14. A method according to claim 2, wherein the ligase is T4 DNA ligase.

15. A method according to claim 3, wherein the ligase is T4 DNA ligase.

15. A method according to claim 4, wherein the ligase is T4 DNA ligase.

16. A method according to claim 5, wherein the ligase is T4 DNA ligase.

17. A method according to claim 9, wherein the ligase is T4 DNA ligase.

18. A method according to claim 10, wherein the ligase is T4 DNA ligase.

19. A method according to claim 11, wherein the ligase is T4 DNA ligase.

20. A method according to claim 12, wherein the ligase is T4 DNA ligase.

21. A method according to claim 13, wherein the ligase is T4 DNA ligase.
